(19) United States
(12) Patent Application Publication
(10) Pub. No.: US 2026/0014003 A1
(43) Pub. Date: Jan. 15, 2026

(54) Anti-Rotation Liner

(71) Applicant: Alps South, LLC, St. Petersburg, FL (US)

(72) Inventors: Aldo Laghi, Pinellas Park, FL (US); David Carson, Clearwater, FL (US)

(21) Appl. No.: 19/265,781

(22) Filed: Jul. 10, 2025

Related U.S. Application Data
(60) Provisional application No. 63/669,782, filed on Jul. 11, 2024.

(51) Int. Cl. A61F 2/78; A61F 2/76

(52) U.S. Cl. CPC A61F 2/7812; A61F 2/76; A61F 2002/7818

(57) ABSTRACT

The present disclosure provides a prosthetic liner comprising a liner body configured to fit over a residual limb of an amputee, the liner body having an outer surface, and one or more elastomeric regions disposed on the outer surface of the liner body. The one or more elastomeric regions are configured to increase friction between the outer surface of the liner body and an inner surface of a prosthetic socket. The elastomeric regions may be arranged in a pattern on the outer surface, such as strips, lines, dots, or a customizable image. The liner body may be covered with a fabric material except for the elastomeric regions, which are formed from an exposed elastic polymer material.

### CROSS-REFERENCE TO RELATED APPLICATIONS

This application claims priority to U.S. Application No. 63/669,782, titled “Anti-Rotation Liner,” filed Jul. 11, 2024, which is hereby incorporated by reference in its entirety.

### FIELD OF INVENTION

The present disclosure relates to prosthetic liners for amputees, and more particularly to an anti-rotation prosthetic liner with friction-enhancing elements designed to prevent rotation within a prosthetic socket.

### BACKGROUND

Prosthetic liners serve as an interface between an amputee's residual limb and a prosthetic socket. These liners provide cushioning, protection, and comfort for the residual limb while also helping to secure the prosthesis to the body. Traditionally, prosthetic liners have been made from materials such as silicone, thermoplastic elastomers, or polyurethane.

One challenge faced by many amputees is rotation of the prosthetic liner within the socket during use. This rotation can occur due to the forces exerted on the prosthesis during walking and other activities. Rotation of the liner may lead to discomfort, skin irritation, and reduced control of the prosthesis.

Various approaches have been explored to address liner rotation. Some liners incorporate textured outer surfaces or geometric patterns to increase friction between the liner and socket. Others utilize suction or vacuum suspension systems to help hold the liner in place. However, these methods may not fully prevent rotation in all cases, particularly for users with conical or symmetrical residual limbs.

The fit and function of a prosthetic liner can be affected by changes in limb volume throughout the day. As an amputee's residual limb fluctuates in size due to factors like activity level and fluid retention, gaps may form between the liner and socket. These gaps can exacerbate rotation issues.

Prosthetic liners typically feature a fabric outer covering to increase durability and facilitate donning and doffing. This fabric layer also helps control the longitudinal stretch of the liner. However, the smooth nature of many fabric coverings may contribute to rotation within the socket.

Advancements in prosthetic liner design continue to be pursued to enhance comfort, fit, and overall function for amputees. Addressing rotation while maintaining other beneficial properties of liners remains an area of ongoing research and development in the field of prosthetics.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, an anti-rotational liner for a prosthetic device is provided. The anti-rotational liner includes a liner body configured to fit over a residual limb of an amputee. The liner body has an outer surface that interfaces with an inner surface of a prosthetic socket. The anti-rotational liner further includes one or more elastomeric regions disposed on the outer surface of the liner body. These elastomeric regions are configured to increase friction between the outer surface of the liner body and the inner surface of the prosthetic socket, thereby reducing rotation of the prosthetic socket relative to the residual limb during use.

According to other aspects of the present disclosure, the anti-rotational liner may include one or more of the following features. The one or more elastomeric regions may be arranged in a pattern on the outer surface of the liner body. The pattern may comprise strips, lines, dots, or a customizable image or logo. The one or more elastomeric regions may include a medial pattern and a lateral pattern disposed on opposite sides of the liner body. The one or more elastomeric regions may include an anterior patch disposed on an anterior side of the liner body. The liner body may be covered with a fabric material except for the one or more elastomeric regions. The one or more elastomeric regions may be formed from an elastic polymer material that is exposed on the outer surface of the liner body.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates an orthogonal view of a prosthetic liner with a friction strip with a partial cross-section to show the internal elastomer layer, according to aspects of the present disclosure.

FIG. 2 depicts an orthogonal view of a prosthetic liner with circular elements with a partial cross-section to show the internal elastomer layer, according to an embodiment.

FIG. 3 shows an orthogonal view of a prosthetic liner with rectangular patches arranged vertically with a partial cross-section to show the internal elastomer layer, according to aspects of the present disclosure.

FIG. 4 illustrates an orthogonal view of an anti-rotation liner with text labeling with a partial cross-section to show the internal elastomer layer, according to an embodiment.

FIG. 5 depicts an orthogonal view of a prosthetic liner featuring wavy lines on its outer surface with a partial cross-section to show the internal elastomer layer, according to aspects of the present disclosure.

FIG. 6 shows an orthogonal view of a prosthetic liner with a vertical strip design with a partial cross-section to show the internal elastomer layer, according to an embodiment.

FIG. 7 illustrates an orthogonal view of a prosthetic liner with circular and rectangular elements with a partial cross-section to show the internal elastomer layer, according to aspects of the present disclosure.

FIG. 8 depicts an orthogonal view of an anti-rotation liner component with text markings with a partial cross-section to show the internal elastomer layer, according to an embodiment.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The prosthetic liner 10 may include an open proximal end 12 and a closed distal end 14. In some cases, the prosthetic liner 10 comprises an external fabric layer 16 and an internal elastomeric layer 18. The prosthetic liner 10 may be configured to provide an interface between a residual limb and a prosthetic socket.

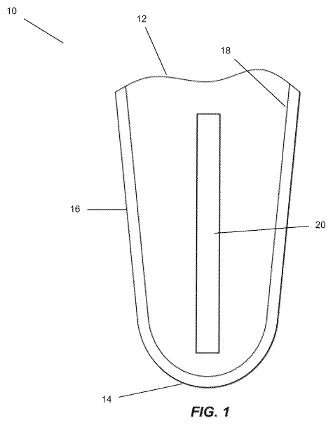

As shown in FIG. 1, the prosthetic liner 10 may have a generally cylindrical shape with tapered sides. The open proximal end 12 may be wider than the closed distal end 14, allowing the prosthetic liner 10 to conform to the anatomical contours of a residual limb.

In some implementations, the prosthetic liner 10 incorporates areas of exposed elastomeric material on the external fabric layer 16. These areas may increase friction between the prosthetic liner 10 and the socket walls. For example, FIG. 1 illustrates a vertical strip positioned centrally along the length of the prosthetic liner 10, which may represent an area of exposed elastomeric material.

The external fabric layer 16 may cover a majority of the prosthetic liner 10, while the areas of exposed elastomeric material 20 may be selectively positioned to provide anti-rotation functionality. This configuration may allow for controlled friction zones while still enabling proper donning and doffing of the prosthetic liner 10.

The internal elastomeric layer 18 may provide cushioning and comfort for the residual limb. In some cases, the internal elastomeric layer 18 may extend throughout the interior of the prosthetic liner 10, from the open proximal end 12 to the closed distal end 14.

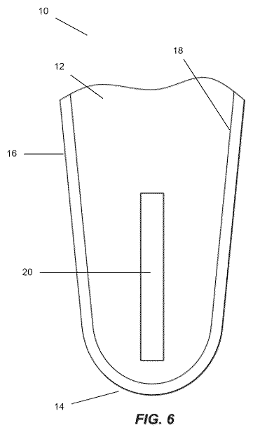

The prosthetic liner 10 may incorporate a vertical strip design as shown in FIG. 1 and FIG. 6. This vertical strip may represent an area of exposed elastomeric material on the external surface of the prosthetic liner 10. In some cases, the vertical strip extends along the length of the prosthetic liner 10, from near the open proximal end 12 towards the closed distal end 14, and in other cases may only extend a portion of the prosthetic liner 10, as shown in FIG. 6.

The vertical strip may be positioned centrally on the external surface of the prosthetic liner 10. In some implementations, the width of the vertical strip may be a fraction of the overall circumference of the prosthetic liner 10. For example, the width of the vertical strip may range from about 5% to about 25% of the circumference of the prosthetic liner 10 at its widest point.

The exposed elastomeric material of the vertical strip may increase friction between the prosthetic liner 10 and the socket walls. This increased friction may help prevent rotation of the prosthetic liner 10 within the socket during use. The vertical orientation of the strip may allow for controlled friction along the entire length of the prosthetic liner 10.

In some cases, the vertical strip may be formed by selectively removing portions of the external fabric layer 16 to expose the underlying internal elastomeric layer 18. Alternatively, the vertical strip may be a raised gel portion 20 applied to the external surface of the prosthetic liner 10.

The placement of the vertical strip may allow for easier donning and doffing of the prosthetic liner 10 compared to a fully exposed elastomeric surface. The external fabric layer 16 may remain intact on the areas of the prosthetic liner 10 not covered by the vertical strip, maintaining the overall durability and stretch characteristics of the prosthetic liner 10.

In some implementations, the raised gel portion 20 may take forms other than a vertical strip. For example, the raised gel portion 20 may be configured as lines, dots, or a customizable image or logo on the external surface of the prosthetic liner 10. These alternative configurations may provide similar anti-rotation functionality while allowing for customization or branding of the prosthetic liner 10.

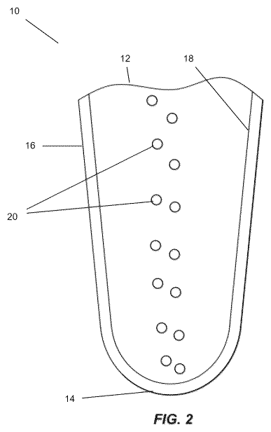

The prosthetic liner 10 may incorporate a circular pattern anti-rotation feature as illustrated in FIG. 2. In some cases, the prosthetic liner 10 includes a series of circular elements arranged in a vertical pattern along the central region of the external surface. These circular elements may be configured as raised gel portions 20 on the external fabric layer 16 of the prosthetic liner 10.

The circular elements may be arranged in a slightly curved configuration, following the contour of the prosthetic liner 10 surface. In some implementations, the circular elements may be uniformly spaced along the length of the prosthetic liner 10, from near the open proximal end 12 towards the closed distal end 14.

The raised gel portions 20 forming the circular elements may increase friction between the prosthetic liner 10 and the socket walls. This increased friction may help prevent rotation of the prosthetic liner 10 within the socket during use. The circular shape of the elements may allow for multidirectional resistance to rotational forces, potentially providing enhanced stability compared to a single vertical strip design.

In some cases, the diameter of the circular elements may range from about 5% to about 15% of the circumference of the prosthetic liner 10 at its widest point. The spacing between the circular elements may be adjusted to provide optimal anti-rotation performance while still allowing for case of donning and doffing.

The circular pattern design may offer advantages over the vertical strip design in certain applications. For example, the discrete nature of the circular elements may allow for greater flexibility in the prosthetic liner 10, potentially improving comfort for the user. Additionally, the circular pattern may distribute the anti-rotation forces more evenly across the surface of the prosthetic liner 10, which may reduce the risk of localized pressure points or irritation.

In some implementations, the raised gel portions 20 forming the circular elements may be integrally formed with the internal elastomeric layer 18, extending through openings in the external fabric layer 16. Alternatively, the raised gel portions 20 may be separately applied to the external surface of the prosthetic liner 10.

The arrangement of the circular elements may be customized based on the specific needs of the user or the characteristics of the residual limb. For instance, the size, spacing, or number of circular elements may be adjusted to provide optimal anti-rotation performance for different limb shapes or activity levels.

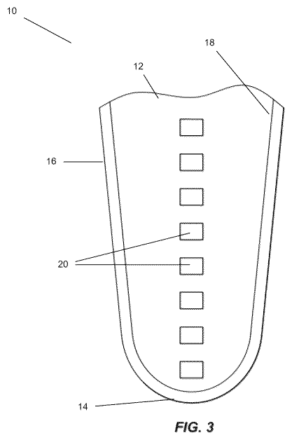

The prosthetic liner 10 may incorporate a plurality of rectangular patches as illustrated in FIG. 3. In some cases, the prosthetic liner 10 includes a series of rectangular patches or areas arranged in a vertical column along the central portion of the external fabric layer 16. These rectangular patches may be configured as raised gel portions 20 on the external fabric layer 16 of the prosthetic liner 10.

The rectangular patches may be uniformly spaced and sized along the length of the prosthetic liner 10, extending from near the open proximal end 12 towards the closed distal end 14. In some implementations, the number of rectangular patches may vary depending on the size and intended use of the prosthetic liner 10. For example, FIG. 3 shows approximately eight distinct rectangular sections visible on the external surface.

Each rectangular patch may have dimensions that are a fraction of the overall circumference and length of the prosthetic liner 10. In some cases, the width of each rectangular patch may range from about 10% to about 30% of the circumference of the prosthetic liner 10 at its widest point. The height of each rectangular patch may range from about 5% to about 15% of the total length of the prosthetic liner 10.

The spacing between the rectangular patches may be designed to provide optimal anti-rotation performance while still allowing for case of donning and doffing. In some implementations, the vertical gap between adjacent rectangular patches may be approximately equal to or slightly larger than the height of each patch.

The raised gel portions 20 forming the rectangular patches may increase friction between the prosthetic liner 10 and the socket walls. This increased friction may help prevent rotation of the prosthetic liner 10 within the socket during use. The rectangular shape and vertical arrangement of the patches may provide resistance to rotational forces along the entire length of the prosthetic liner 10.

In some cases, the raised gel portions 20 forming the rectangular patches may be integrally formed with the internal elastomeric layer 18, extending through openings in the external fabric layer 16. Alternatively, the raised gel portions 20 may be separately applied to the external surface of the prosthetic liner 10.

The arrangement of rectangular patches may offer advantages in certain applications. For example, the discrete nature of the patches may allow for greater flexibility in the prosthetic liner 10 compared to a continuous vertical strip design. Additionally, the spaces between the patches may facilitate easier donning and doffing of the prosthetic liner 10, as the external fabric layer 16 remains intact in these areas.

The rectangular patch design may also allow for customization based on the specific needs of the user or the characteristics of the residual limb. For instance, the size, spacing, or number of rectangular patches may be adjusted to provide optimal anti-rotation performance for different limb shapes or activity levels.

In some implementations, the rectangular patches may be arranged in multiple vertical columns around the circumference of the prosthetic liner 10. This configuration may provide enhanced anti-rotation properties by increasing the total surface area of exposed elastomeric material while maintaining the benefits of discrete patches.

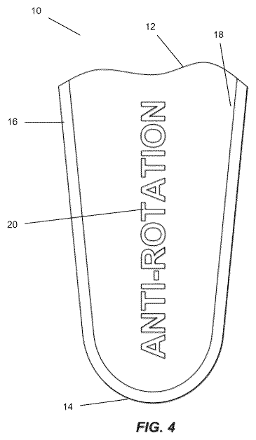

The prosthetic liner 10 may incorporate a text-based anti-rotation feature as illustrated in FIG. 4. For example, the prosthetic liner 10 includes text reading “ANTI-ROTATION” vertically along the external surface. This text may be configured as a raised gel portion 20 on the external fabric layer 16 of the prosthetic liner 10. Any text may be used in this embodiment of the present invention.

The text-based anti-rotation feature may extend from near the open proximal end 12 towards the closed distal end 14 of the prosthetic liner 10. In some implementations, the text may be positioned centrally on the external surface of the prosthetic liner 10, following the contour of the liner's tapered profile.

The raised gel portion 20 forming the “ANTI-ROTATION” text may increase friction between the prosthetic liner 10 and the socket walls. This increased friction may help prevent rotation of the prosthetic liner 10 within the socket during use. The vertical orientation of the text may provide resistance to rotational forces along the entire length of the prosthetic liner 10.

In some cases, the height of the text characters may range from about 5% to about 15% of the total length of the prosthetic liner 10. The width of the text may be a fraction of the overall circumference of the prosthetic liner 10, for example, ranging from about 20% to about 40% of the circumference at its widest point.

The raised gel portion 20 forming the text may be integrally formed with the internal elastomeric layer 18, extending through openings in the external fabric layer 16. Alternatively, the raised gel portion 20 may be separately applied to the external surface of the prosthetic liner 10.

The text-based design may offer unique advantages in certain applications. For example, the text may serve a dual purpose of providing anti-rotation functionality while also clearly identifying the purpose of the prosthetic liner 10. This may be beneficial for users, caregivers, or medical professionals in quickly identifying the specialized nature of the prosthetic liner 10.

In some implementations, the text-based design may allow for customization based on the specific needs of the user or the characteristics of the residual limb. For instance, the size, font, or specific wording of the text may be adjusted to provide optimal anti-rotation performance while maintaining clear legibility.

The spaces between the characters in the “ANTI-ROTATION” text may facilitate easier donning and doffing of the prosthetic liner 10, as the external fabric layer 16 may remain intact in these areas. This configuration may provide a balance between anti-rotation functionality and case of use.

In some cases, the text-based anti-rotation feature may be combined with other anti-rotation designs, such as additional raised gel portions 20 in the form of dots, lines, or patterns as shown in the other figures of the present invention. This combination may enhance the overall anti-rotation performance of the prosthetic liner 10 while maintaining the informative aspect of the text-based design.

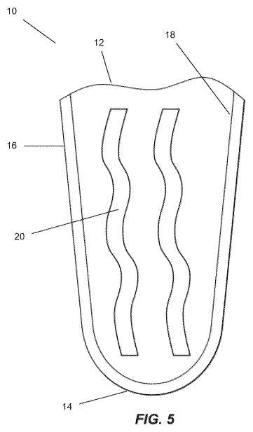

The prosthetic liner 10 may incorporate a wavy line anti-rotation feature as illustrated in FIG. 5. In some cases, the prosthetic liner 10 includes at least two parallel wavy or undulating lines that extend vertically along the length of the external surface. These wavy lines may be configured as raised gel portions 20 on the external fabric layer 16 of the prosthetic liner 10.

The wavy lines may extend from near the open proximal end 12 towards the closed distal end 14 of the prosthetic liner 10. In some implementations, the wavy lines may be positioned centrally on the external surface of the prosthetic liner 10, maintaining consistent spacing between them along their entire length.

The raised gel portions 20 forming the wavy lines may increase friction between the prosthetic liner 10 and the socket walls. This increased friction may help prevent rotation of the prosthetic liner 10 within the socket during use. The undulating pattern of the lines may provide resistance to rotational forces in multiple directions, potentially enhancing the anti-rotation properties compared to straight vertical lines.

In some cases, the amplitude of the waves in the wavy lines may range from about 2% to about 10% of the circumference of the prosthetic liner 10 at its widest point. The wavelength of the undulations may be adjusted to provide optimal anti-rotation performance while maintaining flexibility of the prosthetic liner 10.

The spacing between the two wavy lines may be designed to provide a balance between anti-rotation functionality and case of donning and doffing. In some implementations, the gap between the wavy lines may range from about 10% to about 30% of the circumference of the prosthetic liner 10 at its widest point.

The raised gel portions 20 forming the wavy lines may be integrally formed with the internal elastomeric layer 18, extending through openings in the external fabric layer 16. Alternatively, the raised gel portions 20 may be separately applied to the external surface of the prosthetic liner 10.

The wavy line design may offer advantages in certain applications. For example, the undulating pattern may allow for greater flexibility in the prosthetic liner 10 compared to straight vertical lines or rectangular patches. This increased flexibility may potentially improve comfort for the user while still providing effective anti-rotation properties.

In some implementations, the wavy line pattern may distribute the anti-rotation forces more evenly across the surface of the prosthetic liner 10. This distribution may reduce the risk of localized pressure points or irritation that could occur with more rigid anti-rotation features.

The areas of the prosthetic liner 10 not covered by the wavy lines may retain the properties of the external fabric layer 16, potentially facilitating easier donning and doffing compared to a fully exposed elastomeric surface. This configuration may provide a balance between anti-rotation functionality and user comfort.

In some cases, the specific pattern of the wavy lines may be customized based on the needs of the user or the characteristics of the residual limb. For instance, the amplitude, wavelength, or number of wavy lines may be adjusted to provide optimal anti-rotation performance for different limb shapes or activity levels.

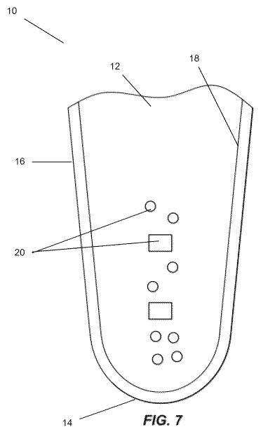

The prosthetic liner 10 may incorporate a mixed geometric pattern anti-rotation feature as illustrated in FIG. 7. In some cases, the prosthetic liner 10 includes a specific arrangement of geometric elements, including circles and rectangles positioned in a vertical alignment along the central portion of the external surface.

The mixed geometric pattern may consist of several circular elements interspersed with rectangular elements, creating a systematic arrangement down the length of the prosthetic liner 10. This pattern may extend from near the open proximal end 12 towards the closed distal end 14.

In some implementations, the circular and rectangular elements may be configured as raised gel portions 20 on the external fabric layer 16 of the prosthetic liner 10. These raised gel portions 20 may increase friction between the prosthetic liner 10 and the socket walls, potentially enhancing the anti-rotation properties of the prosthetic liner 10.

The arrangement of the mixed geometric pattern may include alternating circular and rectangular elements. For example, the pattern may begin with a circular element near the open proximal end 12, followed by a rectangular element, then another circular element, and so on. In some cases, the pattern may conclude with a circular element near the closed distal end 14.

The size of the circular elements may range from about 5% to about 15% of the circumference of the prosthetic liner 10 at its widest point. The rectangular elements may have a width ranging from about 10% to about 20% of the circumference and a height ranging from about 5% to about 10% of the total length of the prosthetic liner 10.

The spacing between the geometric elements may be designed to provide optimal anti-rotation performance while still allowing for case of donning and doffing. In some implementations, the vertical gap between adjacent elements may be approximately equal to or slightly larger than the height of the rectangular elements.

The use of multiple shapes in the anti-rotation design may offer certain advantages. For example, the combination of circular and rectangular elements may provide resistance to rotational forces in multiple directions. The circular elements may offer omnidirectional resistance, while the rectangular elements may provide enhanced resistance along their longer edges.

In some cases, the mixed geometric pattern may distribute the anti-rotation forces more evenly across the surface of the prosthetic liner 10 compared to a single-shape design. This distribution may potentially reduce the risk of localized pressure points or irritation for the user.

The raised gel portions 20 forming the mixed geometric pattern may be integrally formed with the internal elastomeric layer 18, extending through openings in the external fabric layer 16. Alternatively, the raised gel portions 20 may be separately applied to the external surface of the prosthetic liner 10.

The areas of the prosthetic liner 10 not covered by the geometric elements may retain the properties of the external fabric layer 16, potentially facilitating easier donning and doffing compared to a fully exposed elastomeric surface. This configuration may provide a balance between anti-rotation functionality and user comfort.

In some implementations, the specific arrangement, size, or number of circular and rectangular elements in the mixed geometric pattern may be customized based on the needs of the user or the characteristics of the residual limb. This customization may allow for optimization of the anti-rotation performance for different limb shapes or activity levels.

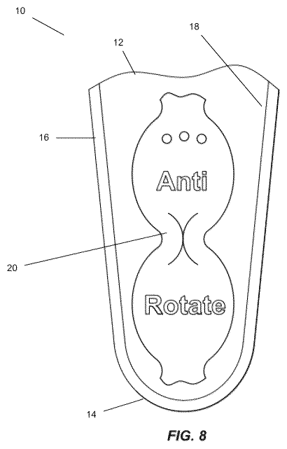

The prosthetic liner 10 may incorporate an hourglass-shaped anti-rotation feature as illustrated in FIG. 8. In some cases, the prosthetic liner 10 includes a curved outer profile that tapers inward at its center, creating an hourglass-like shape. This unique shape may extend from the open proximal end 12 to the closed distal end 14 of the prosthetic liner 10.

The hourglass-shaped design of the prosthetic liner 10 may contribute to preventing rotation within a prosthetic socket. In some implementations, the constricted middle section of the prosthetic liner 10 may provide increased resistance against rotational forces, potentially enhancing the overall stability of the prosthetic fit.

The external surface of the prosthetic liner 10 may include text markings showing “Anti” and “Rotate” separated by the constricted middle section. These text markings may be configured as raised gel portions 20 on the external fabric layer 16 of the prosthetic liner 10. The raised gel portions 20 forming the text may increase friction between the prosthetic liner 10 and the socket walls, further enhancing the anti-rotation properties.

In some cases, the prosthetic liner 10 may include three small circular elements arranged horizontally near the open proximal end 12. These circular elements may also be configured as raised gel portions 20, potentially providing additional points of friction and resistance to rotation.

The entire hourglass-shaped component of the prosthetic liner 10 may be enclosed within a thin border that follows the general contour of the liner while maintaining a slight offset from the liner's edge. This border may represent a transition zone between the anti-rotation features and the standard external fabric layer 16 of the prosthetic liner 10.

The hourglass shape of the prosthetic liner 10 may offer additional functionality beyond anti-rotation properties. In some implementations, the constricted middle section may provide improved flexibility, potentially allowing for easier donning and doffing of the prosthetic liner 10. The wider sections at the open proximal end 12 and closed distal end 14 may provide enhanced stability and comfort for the user.

In some cases, the internal elastomeric layer 18 of the prosthetic liner 10 may be contoured to match the hourglass shape of the external profile. This contouring may help distribute pressure more evenly across the residual limb, potentially improving comfort and reducing the risk of skin irritation.

The combination of the hourglass shape, text markings, and circular elements may create a multifaceted approach to preventing rotation. The shape may provide mechanical resistance, while the raised gel portions 20 forming the text and circular elements may increase friction. This integrated design may offer enhanced anti-rotation performance compared to more traditional liner shapes.

In some implementations, the specific dimensions of the hourglass shape may be customized based on the needs of the user or the characteristics of the residual limb. For instance, the degree of constriction in the middle section or the overall proportions of the prosthetic liner 10 may be adjusted to provide optimal anti-rotation performance for different limb shapes or activity levels.

The prosthetic liner 10 may incorporate a raised gel portion 20 as an anti-rotation feature. In some cases, the raised gel portion 20 may be located on the external surface of the prosthetic liner 10, providing increased friction between the prosthetic liner 10 and the socket walls.

The raised gel portion 20 may be composed of an elastic polymer material. This elastic polymer may enhance the anti-rotation properties of the prosthetic liner 10 by creating a high-friction interface with the socket walls.

In some implementations, the raised gel portion 20 may be arranged in a medial and lateral pattern on either side of the prosthetic liner 10. This configuration may provide balanced anti-rotation properties while allowing for easier donning and doffing compared to a fully exposed patch of elastomeric material.

The raised gel portion 20 may also be configured as a patch on the anterior side of the prosthetic liner 10. This anterior placement may offer targeted anti-rotation functionality in areas where rotational forces are most likely to occur during use.

The external fabric layer 16 of the prosthetic liner 10 may remain intact in areas not covered by the raised gel portion 20. This configuration may facilitate easier donning and doffing of the prosthetic liner 10 compared to a prosthetic liner with a fully exposed elastomeric surface.

In some cases, the raised gel portion 20 may be integrally formed with the internal elastomeric layer 18, extending through openings in the external fabric layer 16. Alternatively, the raised gel portion 20 may be separately applied to the external surface of the prosthetic liner 10.

The size, shape, and placement of the raised gel portion 20 may be customized based on the specific needs of the user or the characteristics of the residual limb. For instance, the pattern or coverage area of the raised gel portion 20 may be adjusted to provide optimal anti-rotation performance for different limb shapes or activity levels.

The raised gel portion 20 may interact with other components of the prosthetic liner 10 to enhance overall performance. For example, the combination of the raised gel portion 20 and the external fabric layer 16 may provide a balance between anti-rotation functionality and user comfort.

In some implementations, the raised gel portion 20 may be designed to maintain flexibility of the prosthetic liner 10 while still providing effective anti-rotation properties. This flexibility may contribute to user comfort and ease of use during daily activities.

The interface between the raised gel portion 20 and the external fabric layer 16 may be designed to ensure durability and longevity of the prosthetic liner 10. In some cases, this interface may be reinforced to prevent separation or degradation over time.

A number of implementations have been described. Nevertheless, it will be understood that various modifications may be made without departing from the spirit and scope of the disclosure. Accordingly, other implementations are within the scope of the following claims.

1. A prosthetic liner, comprising:
a liner body configured to fit over a residual limb of an amputee, the liner body having an outer surface; and
one or more elastomeric regions disposed on the outer surface of the liner body, wherein the one or more elastomeric regions are configured to increase friction between the outer surface of the liner body and an inner surface of a prosthetic socket.

2. The prosthetic liner of claim 1, wherein the one or more elastomeric regions are arranged in a pattern on the outer surface of the liner body.

3. The prosthetic liner of claim 2, wherein the pattern comprises at least one of strips, lines, dots, or a customizable image.

4. The prosthetic liner of claim 1, wherein the one or more elastomeric regions comprise a medial pattern and a lateral pattern disposed on opposite sides of the liner body.

5. The prosthetic liner of claim 1, wherein the one or more elastomeric regions comprise an anterior patch disposed on an anterior side of the liner body.

6. The prosthetic liner of claim 1, wherein the liner body is covered with a fabric material except for the one or more elastomeric regions.

7. The prosthetic liner of claim 1, wherein the one or more elastomeric regions are formed from an elastic polymer material that is exposed on the outer surface of the liner body.

8. The prosthetic liner of claim 1, wherein the liner body comprises an open proximal end and a closed distal end.

9. The prosthetic liner of claim 8, wherein the liner body comprises an external fabric layer and an internal elastomeric layer.

10. A method of manufacturing a prosthetic liner, comprising:
forming a liner body configured to fit over a residual limb of an amputee, the liner body having an outer surface; and
disposing one or more elastomeric regions on the outer surface of the liner body, wherein the one or more elastomeric regions are configured to increase friction between the outer surface of the liner body and an inner surface of a prosthetic socket.

11. The method of claim 10, wherein disposing the one or more elastomeric regions comprises arranging the elastomeric regions in a pattern on the outer surface of the liner body.

12. The method of claim 11, wherein the pattern comprises at least one of strips, lines, dots, or a customizable image.

13. The method of claim 10, wherein disposing the one or more elastomeric regions comprises forming a medial pattern and a lateral pattern on opposite sides of the liner body.

14. The method of claim 10, wherein disposing the one or more elastomeric regions comprises forming an anterior patch on an anterior side of the liner body.

15. The method of claim 10, further comprising covering the liner body with a fabric material except for the one or more elastomeric regions.

16. The method of claim 15, wherein the one or more elastomeric regions are formed from an elastic polymer material that is exposed on the outer surface of the liner body.

17. A prosthetic system, comprising:
a prosthetic socket having an inner surface; and
a prosthetic liner comprising a liner body configured to fit over a residual limb of an amputee, the liner body having an outer surface, and one or more elastomeric regions disposed on the outer surface of the liner body, wherein the one or more elastomeric regions are configured to increase friction between the outer surface of the liner body and the inner surface of the prosthetic socket.

18. The prosthetic system of claim 17, wherein the one or more elastomeric regions are arranged in a pattern on the outer surface of the liner body, the pattern comprising at least one of strips, lines, dots, or a customizable image.

19. The prosthetic system of claim 18, wherein the pattern comprises a medial pattern and a lateral pattern disposed on opposite sides of the liner body.

20. The prosthetic system of claim 19, wherein the liner body is covered with a fabric material except for the one or more elastomeric regions, and wherein the one or more elastomeric regions are formed from an elastic polymer material that is exposed on the outer surface of the liner body.
